(19) United States
(12) Patent Application Publication
(10) Pub. No.: US 2013/0281858 A1
(43) Pub. Date: Oct. 24, 2013

(54) METHOD FOR INCREASING DEPTH OF FIELD AND ULTRASOUND IMAGING SYSTEM USING THE SAME

(71) Applicant: INDUSTRIAL TECHNOLOGY RESEARCH INSTITUTE, Hsinchu (TW)

(72) Inventors: Chu-Yu Huang, Taichung City (TW); Chuan-Chung Chang, Hsinchu County (TW); Hsin-Yueh Sung, New Taipei City (TW); Chir-Weei Chang, Taoyuan County (TW); Kuo-Tung Tiao, Hsinchu County (TW)

(73) Assignee: Industrial Technology Research Institute, Hsinchu (TW)

(21) Appl. No.: 13/717,657

(22) Filed: Dec. 17, 2012

Related U.S. Application Data
(60) Provisional application No. 61/635,305, filed on Apr. 19, 2012.

(51) Int. Cl. A61B 8/14

(52) U.S. Cl. CPC A61B 8/14
USPC 600/443

(57) ABSTRACT

An ultrasound imaging system and methods thereof are provided. A method includes transmitting a plurality of energy signals coded by a first asymmetric phase element toward an object to be imaged, receiving a plurality of echo signals from the object to be imaged, respectively coding the received signals with a second asymmetric phase element, and reconstructing an image data set with an extended depth of field by decoding the received signals. The ultrasound imaging system includes a transmitter transmitting energy signals coded by a first asymmetric phase element toward an object to be imaged, and a receiver receiving echo signals from the object to be imaged, respectively coding the received signals with a second asymmetric phase element, and reconstructing an image data set with an extended depth of field by decoding the received signals.

### CROSS-REFERENCE TO RELATED APPLICATION

This application claims the priority benefits of U.S. provisional application Ser. No. 61/635,305, filed on Apr. 19, 2012. The entirety of the above-mentioned patent applications is hereby incorporated by reference herein and made a part of this specification.

### TECHNICAL FIELD

The disclosure relates to an ultrasound imaging system and methods thereof.

### BACKGROUND

Conventional ultrasound imaging systems have a short depth of field. Ultrasound beams diverge or spread very quickly away from the focus. Therefore, in a pulse-echo medical imaging system, multiple transmissions of beams focused at different depths are needed to increase an effective depth of field. Transmission of a beam must wait until all echoes of a previous beam return, and since the propagation speed of sound in biological soft tissues is limited, multiple transmissions reduce the image frame rate drastically. Moreover, a low frame rate blurs the images of a moving object, such as the heart.

### SUMMARY

The disclosure provides an ultrasound imaging system, including a transmitter and an emitter. The transmitter is adapted to transmit a plurality of energy signals coded by a first asymmetric phase element toward an object to be imaged. The receiver is adapted to receive a plurality of echo signals from the object to be imaged, respectively code the received signals with a second asymmetric phase element, and reconstruct an image data set with an extended depth of field by decoding the received signals.

The disclosure provides an ultrasound imaging system, including a transmitter and an emitter. The transmitter is adapted to transmit a plurality of energy signals coded by an asymmetric phase element toward an object to be imaged. The receiver is adapted to receive a plurality of echo signals from the object to be imaged and reconstruct an image data set with an extended depth of field by decoding the received signals.

The disclosure provides an ultrasound imaging system, including a transmitter and an emitter. The transmitter is adapted to transmit a plurality of energy signals toward an object to be imaged. The receiver is adapted to receive a plurality of echo signals from the object to be imaged, respectively code the received signals with an asymmetric phase element, and reconstruct an image data set with an extended depth of field by decoding the received signals.

The disclosure provides a method for an ultrasound imaging system, the method including the following steps. A plurality of energy signals coded by a first asymmetric phase element are transmitted toward an object to be imaged. A plurality of echo signals from the object to be imaged are received, the received signals are coded with a second asymmetric phase element, and an image data set with an extended depth of field is reconstructed by decoding the received signals.

The disclosure provides a method for an ultrasound imaging system, the method including the following steps. A plurality of energy signals coded by an asymmetric phase element is transmitted toward an object to be imaged. A plurality of echo signals from the object to be imaged is received, and an image data set with an extended depth of field is reconstructed by decoding the received signals.

The disclosure provides a method for an ultrasound imaging system, the method including the following steps. A plurality of energy signals are transmitted toward an object to be imaged. A plurality of echo signals from the object to be imaged is received, the received signals are coded with an asymmetric phase element, and an image data set with an extended depth of field is reconstructed by decoding the received signals.

Several exemplary embodiments accompanied with figures are described in detail below to further describe the disclosure in details.

### BRIEF DESCRIPTION OF THE DRAWINGS

The accompanying drawings are included to provide further understanding, and are incorporated in and constitute a part of this specification. The drawings illustrate exemplary embodiments and, together with the description, serve to explain the principles of the disclosure.

FIG. 1 is a schematic view of an ultrasound imaging system according to an exemplary embodiment.

FIGS. 2A and 2B are schematic views of the transmitter and the receiver in the ultrasound imaging system depicted in FIG. 1 according to an exemplary embodiment.

FIG. 3 is a schematic view of the signal processor depicted in FIG. 2 according to an exemplary embodiment.

FIGS. 4A and 4B are schematic views of the transmitter and the receiver in the ultrasound imaging system depicted in FIG. 1 according to another exemplary embodiment.

FIGS. 5A and 5B are schematic views of the transmitter and the receiver in the ultrasound imaging system depicted in FIG. 1 according to another exemplary embodiment.

FIGS. 6A and 6B are curve diagrams of the time delays of the transmitting and receiving signals due to an asymmetric phase element in a transmitter and a receiver of an ultrasound imaging system according to an exemplary embodiment.

FIG. 7A depicts a synthetic phantom pattern used for simulating the image formation of an ultrasound imaging system according to an exemplary embodiment.

FIG. 7B depicts an ultrasound image from an ultrasound imaging system using a single focal point at 60 mm for both emission and reception without any asymmetric phase elements.

FIG. 7C depicts an ultrasound image using a cubic phase mask for both emission and reception in an ultrasound imaging system according to an exemplary embodiment.

FIG. 8A depicts an intermediate image using a cubic phase mask for both emission and reception in an ultrasound imaging system according to an exemplary embodiment.

FIG. 8B depicts a decoded ultrasound image using a Wiener filter according to an exemplary embodiment.

FIG. 8C depicts a decoded ultrasound image using a Wiener filter with a −6 dB process according to an exemplary embodiment.

FIG. 9 is a curve diagram comparing the penetration depths between an ultrasound imaging system without any asymmetric phase elements and an ultrasound imaging system with a cubic phase mask according to an exemplary embodiment.

FIGS. 10A and 10B are amplitude diagrams comparing the received response of an ultrasound imaging system with and without a cubic phase mask according to an exemplary embodiment.

FIGS. 11A and 11B are images comparing a depth of field of an ultrasound imaging system with a single focus emission and reception and a depth of field of an ultrasound system with an added cubic phase mask to code the transmitting and receiving signals according to an exemplary embodiment.

FIGS. 12A and 12B are images comparing the focal lateral resolutions of an ultrasound imaging system with and without a cubic phase mask according to an exemplary embodiment.

### DETAILED DESCRIPTION OF DISCLOSED EMBODIMENTS

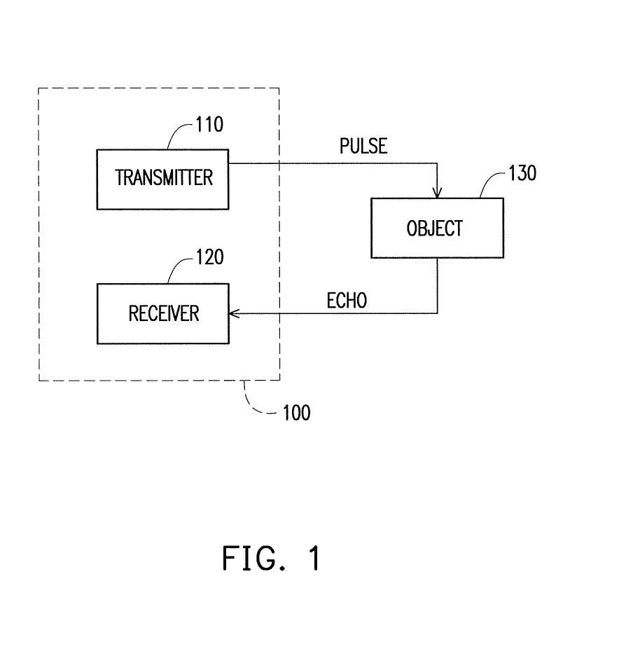

FIG. 1 is a schematic view of an ultrasound imaging system according to an exemplary embodiment. With reference to FIG. 1, an ultrasound imaging system 100 adapted to increase a depth of field for imaging an object 130 may include a transmitter 110 and a receiver 120. The object 130 to be imaged may be an internal organ, for example, although the disclosure is not limited thereto, and other two-dimensional or three-dimensional objects may be imaged by the ultrasound imaging system 100. In some embodiments, the transmitter 110 transmits a plurality of ultrasound signals PULSE toward the object 130 to be imaged, and the receiver 120 is adapted to receive a plurality of echo signals ECHO from the object 130 to be imaged.

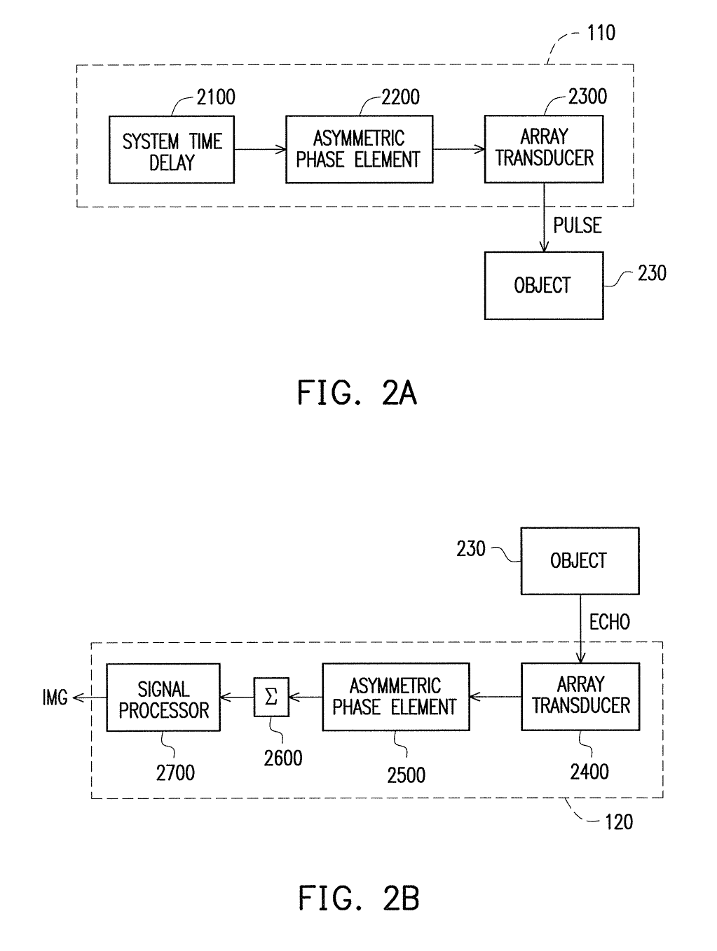

FIGS. 2A and 2B are schematic views of the transmitter and the receiver in the ultrasound imaging system depicted in FIG. 1 according to an exemplary embodiment. Referring to FIG. 2A, in the present embodiment, the transmitter 110 is adapted to transmit a plurality of energy signals coded by a first asymmetric phase element 2200 toward an object 230 to be imaged. According to some embodiments, the transmitter 110 includes a system time delay 2100, the first asymmetric phase element 2200, and an array transducer 2300. In the present embodiment, the system time delay 2100 delays the energy signals, the first asymmetric phase element 2200 codes the delayed energy signals, and the array transducer 2300 converts the delayed energy signals into a plurality of ultrasound signals and respectively transmits the coded ultrasound signals PULSE toward the object 230 to be imaged. It should be noted that, the energy signals delayed by the system time delay 2100 may be generated by a power source or may be applied to the ultrasound imaging system 100 by a driving device (not drawn).

With reference to FIG. 2B, the receiver 120 is adapted to receive a plurality of echo signals ECHO from the object 230 to be imaged, respectively code the received signals with a second asymmetric phase element 2500, and reconstruct an image data set IMG with an extended depth of field by decoding the received signals. In some embodiments, the receiver 120 includes an array transducer 2400, the second asymmetric phase element 2500, a signal adder 2600, and a signal processor 2700. The array transducer 2400 converts each of the echo signals ECHO into a plurality of electrical (e.g. voltage) signals. The second asymmetric phase element 2500 codes the electrical signal, and the signal adder 2600 sums the coded electrical signals into a radio frequency (RF) signal. The signal processor 2700 then combines the RF signals into an intermediate image and decodes the intermediate image into a decoded ultrasound image IMG, as shown in FIG. 2B.

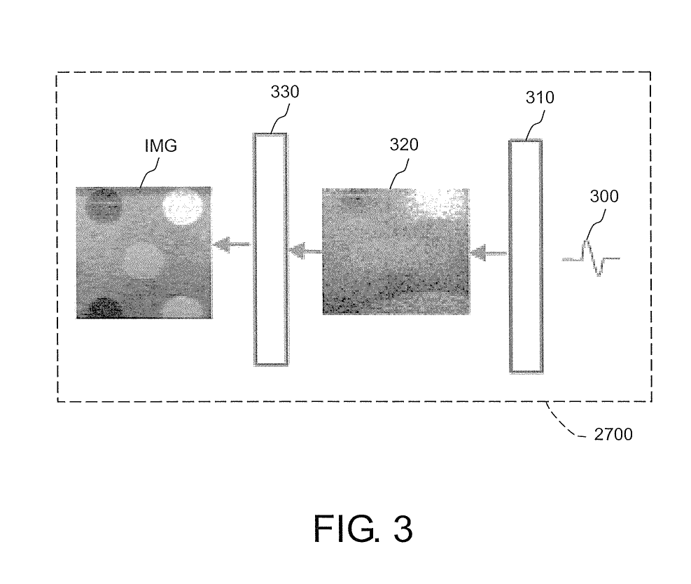

FIG. 3 is a schematic view of the signal processor depicted in FIG. 2 according to an exemplary embodiment. In the present embodiment, the signal processor 2700 includes a RF signal combiner 310 and a decoding filter 330. The RF signal combiner 310 combines the RF signals to form an intermediate image 320. The intermediate image 320 may be an ultrasound image, for example. The decoding filter 330 decodes the intermediate image 320 into the decoded ultrasound image IMG. The decoding filter 330 may be a digital decoding filter, although embodiments of the disclosure are not limited thereto, and the decoding filter 330 may be analog, digital, or implemented by software with a computer running a program having a decoding filter function according to an application. Moreover, the first asymmetric phase element 2200 in FIG. 2A and the second asymmetric phase element 2500 in FIG. 2B may be an asymmetric phase mask, an asymmetric phase function, an asymmetric delay time table, or an asymmetric phase surface integrated with a lens according to an application. Accordingly, the first asymmetric phase element 2200 in FIG. 2A and the second asymmetric phase element 2500 may be implemented by hardware or software. In addition, it should be noted that, when suitable for an application, either the first asymmetric phase element 2200 in FIG. 2A or the second asymmetric phase element 2500 in FIG. 2B may be omitted in the ultrasound imaging system 100.

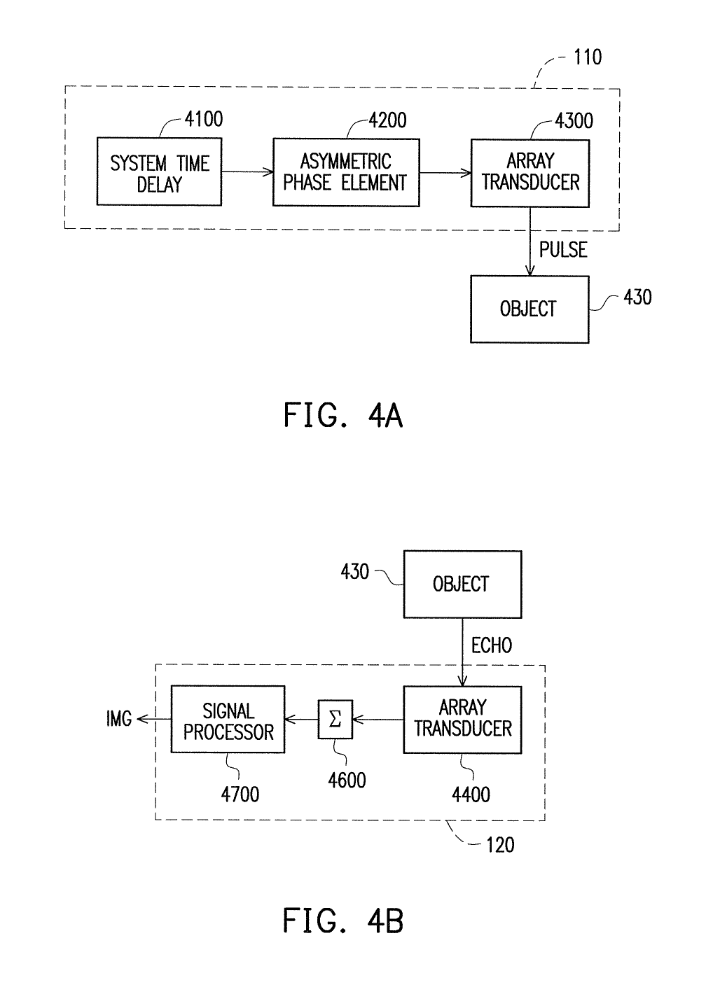

FIGS. 4A and 4B are schematic views of the transmitter and the receiver in the ultrasound imaging system depicted in FIG. 1 according to another exemplary embodiment. Compared with the ultrasound imaging system depicted in FIGS. 2A and 2B, a difference in the ultrasound imaging system shown in FIGS. 4A and 4B is that the asymmetric phase element is omitted in the receiver 120. Referring to FIG. 2A, in the present embodiment, the transmitter 110 is adapted to transmit a plurality of energy signals coded by an asymmetric phase element 4200 toward an object 430 to be imaged. According to some embodiments, the transmitter 110 includes a system time delay 4100, the asymmetric phase element 4200, and an array transducer 4300. In the present embodiment, the system time delay 4100 delays the energy signals, the asymmetric phase element 4200 codes the delayed energy signals, and the array transducer 4300 converts the delayed energy signals into a plurality of ultrasound signals and respectively transmits the coded ultrasound signals PULSE toward the object 430 to be imaged. It should be noted that, the energy signals delayed by the system time delay 4100 may be generated by a power source or may be applied to the ultrasound imaging system 100 by a driving device (not drawn).

With reference to FIG. 4B, the receiver 120 is adapted to receive a plurality of echo signals ECHO from the object 430 to be imaged, and reconstruct an image data set IMG with an extended depth of field by decoding the received signals. In some embodiments, the receiver 120 includes an array transducer 4400, a signal adder 4600, and a signal processor 4700. The array transducer 4400 converts each of the echo signals ECHO into a plurality of electrical (e.g. voltage) signals. The signal adder 4600 sums the electrical signals into a radio frequency (RF) signal. Similar to the signal processor 2700 shown in FIG. 2B, the signal processor 4700 then combines the RF signals into an intermediate image and decodes the intermediate image into a decoded ultrasound image IMG, for example.

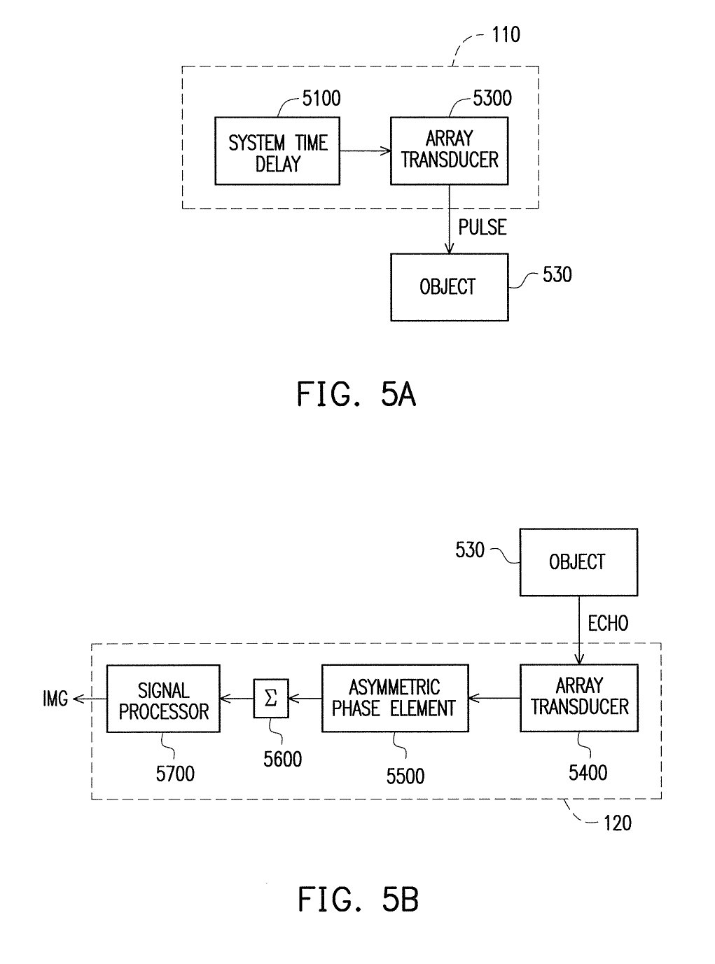

FIGS. 5A and 5B are schematic views of the transmitter and the receiver in the ultrasound imaging system depicted in FIG. 1 according to an exemplary embodiment. Compared with the ultrasound imaging system depicted in FIGS. 2A and 2B, a difference in the ultrasound imaging system shown in FIGS. 5A and 5B is that the asymmetric phase element is omitted in the transmitter 110. Referring to FIG. 5A, in the present embodiment, the transmitter 110 is adapted to transmit a plurality of energy signals toward an object 530 to be imaged. According to some embodiments, the transmitter 110 includes a system time delay 5100 and an array transducer 5300. In the present embodiment, the system time delay 5100 delays the energy signals, and the array transducer 5300 converts the delayed energy signals into a plurality of ultrasound signals and respectively transmits the ultrasound signals PULSE toward the object 530 to be imaged. It should be noted that, the energy signals delayed by the system time delay 4100 may be generated by a power source or may be applied to the ultrasound imaging system 100 by a driving device (not drawn).

With reference to FIG. 5B, the receiver 120 is adapted to receive a plurality of echo signals ECHO from the object 530 to be imaged, respectively code the received signals with an asymmetric phase element 5500, and reconstruct an image data set IMG with an extended depth of field by decoding the received signals. In some embodiments, the receiver 120 includes an array transducer 5400, the asymmetric phase element 5500, a signal adder 5600, and a signal processor 5700. The array transducer 5400 converts each of the echo signals ECHO into a plurality of electrical (e.g. voltage) signals. The asymmetric phase element 5500 codes the electrical signal, and the signal adder 5600 sums the coded electrical signals into a radio frequency (RF) signal. Similar to the signal processor 2700 shown in FIG. 2B, the signal processor 5700 then combines the RF signals into an intermediate image and decodes the intermediate image into a decoded ultrasound image IMG, for example.

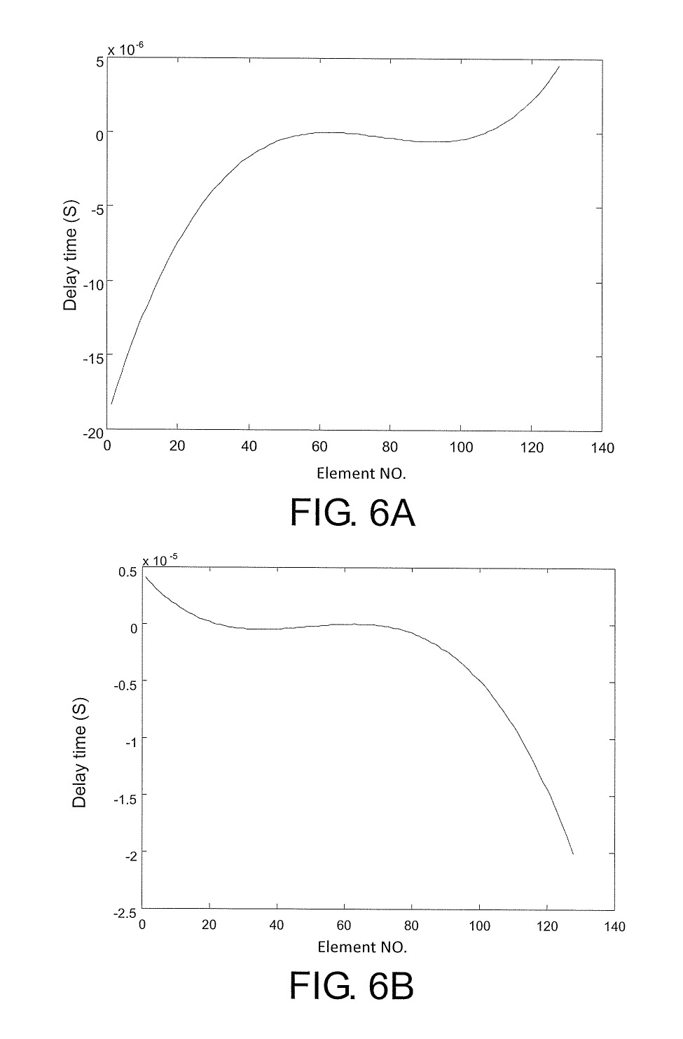

The addition of the first asymmetric phase element 2200 in the transmitter 110 and the second asymmetric phase element 2500 in the receiver 120 as shown in FIGS. 2A and 2B can be simulated for comparison with an ultrasound imaging system without the asymmetric phase elements. FIGS. 6A and 6B are curve diagrams of the time delays of the transmitting and receiving signals due to an asymmetric phase element in a transmitter and a receiver of an ultrasound imaging system according to an exemplary embodiment. In an example for illustrative purposes, a cubic phase mask is used to simulate the asymmetric phase elements 2200 and 2500. For a normalized coordinate x in a linear array transducer used to describe the position of a transducer element relating to the center axis of a transmitting ultrasound beam, the cubic phase mask is defined as P(x) shown in equation (1):

$$P ( x ) = \{ \begin{matrix}α {x}^{3} , & for \vert x \vert \leq 1 \\ 0 , & otherwise\end{matrix} \qquad ( 1 )$$

in which α is parameter used to adjust a depth of field increase. It should be appreciated that, if a two-dimensional array transducer is used, then a two-dimensional P(x,y) can be used to simulate the asymmetric phase element.

As shown in FIGS. 6A and 6B, the transmitting and receiving time delays including the system time delays are simulated. In FIG. 6B, a −1 factor is multiplied to the asymmetric phase element in the receiver to produce a symmetric beam forming result, although in other embodiments symmetric beam formation may not be required. The simulation example may be performed in a Field II program on a computing platform, for example, although the disclosure is not limited thereto.

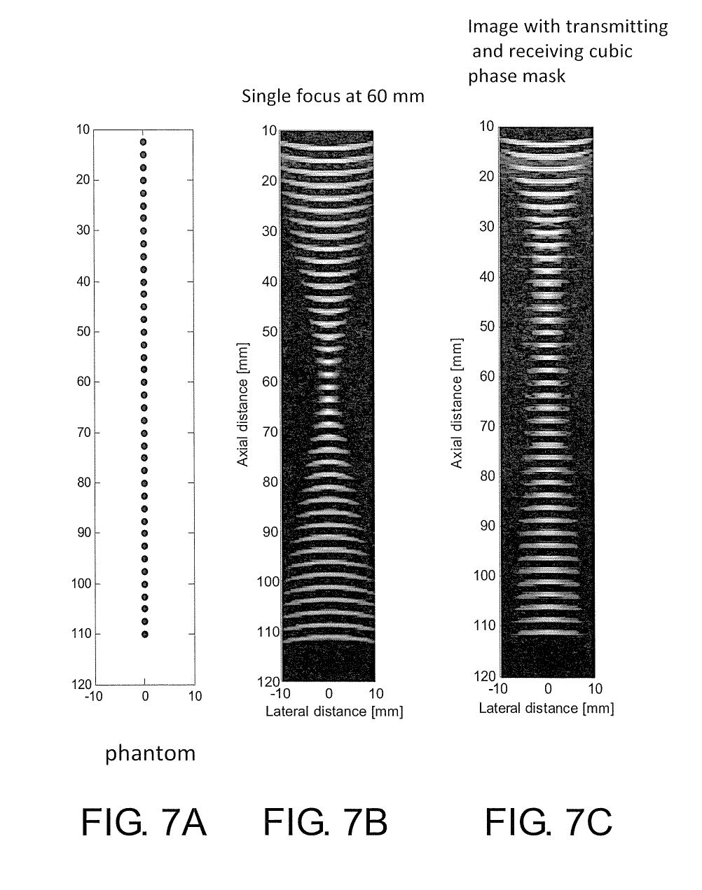

In the illustrative simulation example, a 128 elements array transducer with a nominal frequency of 3 MHz is used. 64 of the transducer elements were used for imaging, and scanning was done by translating the 64 active elements over the aperture and focusing in the proper points. FIG. 7A depict a synthetic phantom pattern used for simulating the image formation of an ultrasound imaging system according to an exemplary embodiment. The synthetic phantom pattern used in FIG. 7A consists of a number of point targets placed with a distance of 2.5 mm starting at 15 mm from the transducer surface. A linear sweep image of the points is then made and the resulting image is compressed to show a 40 dB dynamic range. The results are shown in FIGS. 7B and 7C. FIG. 7B depicts an ultrasound image from an ultrasound imaging system using a single focal point at 60 mm for both emission and reception without any asymmetric phase elements. On the other hand, FIG. 7C depicts an ultrasound image using a cubic phase mask for both emission and reception in an ultrasound imaging system according to an exemplary embodiment. It should be noted that FIG. 7C simulates the intermediate image 320 of FIG. 3 before a decoding process.

In the illustrative simulation example, a Wiener filter is used to form the decoded ultrasound image IMG (e.g. a final image) depicted in FIG. 2B. In the example, an effect of inverse filter in the frequency space can be expressed as:

$$\{ \begin{matrix}final \\ image\end{matrix} \} = ( \{ \begin{matrix}original \\ object\end{matrix} \} \times {H}_{system} ) \times \frac{{H}_{system}^{*}}{μ + {\vert {H}_{system} \vert}^{2}} \qquad ( 2 )$$

Moreover, for Gaussian noise and image statistics, an optimum parameter is:

$${μ}_{optimum} = \frac{1}{{SNR}_{power}} \qquad ( 3 )$$

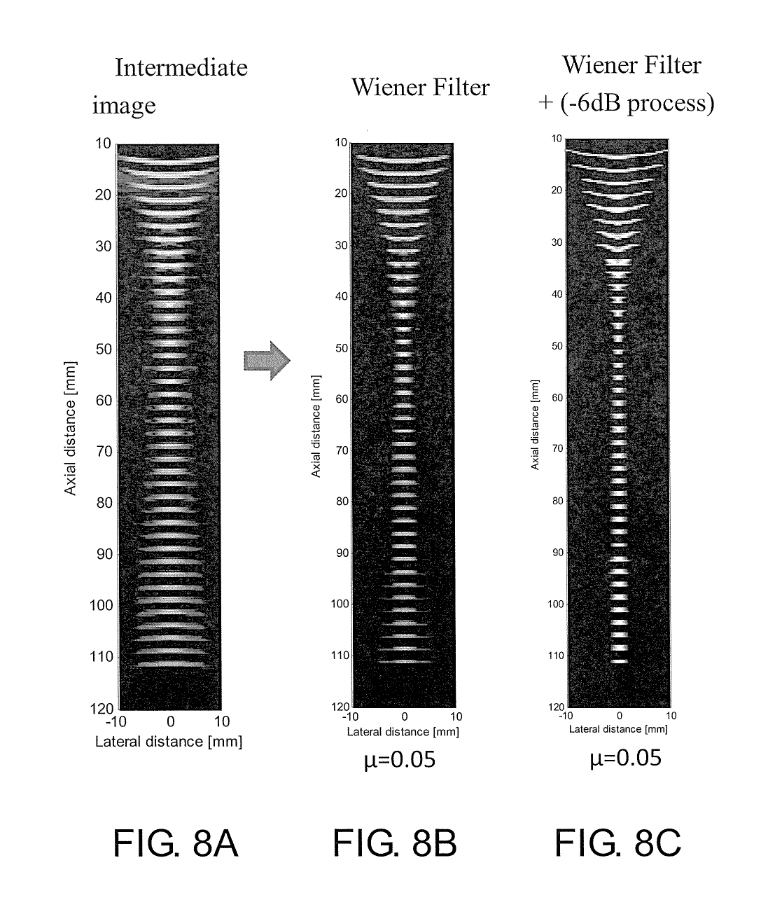

The simulated results of applying a Wiener filter as the decoding filter 330 in FIG. 3 to form the decoded ultrasound image can be observed in FIGS. 8A-8C. FIG. 8A depicts an intermediate image using a cubic phase mask for both emission and reception in an ultrasound imaging system according to an exemplary embodiment. FIG. 8B depicts a decoded ultrasound image using a Wiener filter according to an exemplary embodiment, and FIG. 8C depicts a decoded ultrasound image using a Wiener filter with a −6 dB process according to an exemplary embodiment, in which the parameter μ=0.05 in FIGS. 8B and 8C.

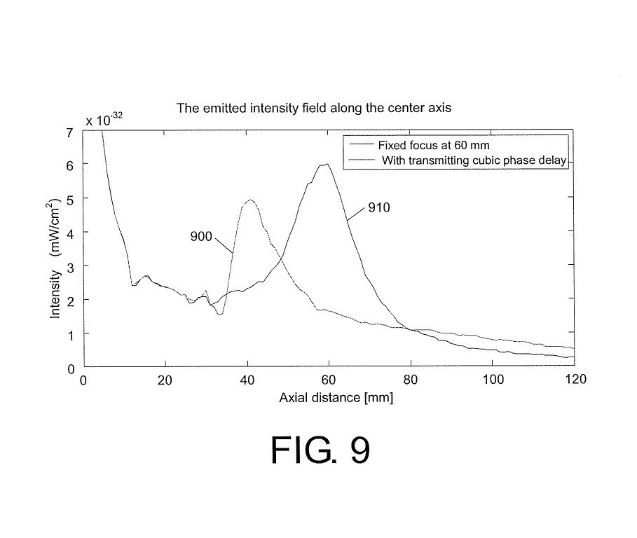

The effect of the asymmetric phase elements to the penetration depth of an ultrasound beam in an ultrasound image system can be observed in the simulated example. FIG. 9 is a curve diagram comparing the penetration depths between an ultrasound imaging system without any asymmetric phase elements and an ultrasound imaging system with a cubic phase mask according to an exemplary embodiment. In FIG. 9, a curve 900 represents an emitted intensity field for only a single focal point at 60 mm without any asymmetric phase elements. On the other hand, a curve 910 represents an emitted intensity field with a cubic phase mask as the asymmetric phase element. As shown in FIG. 9, although the addition of a cubic phase mask results in a lower maximum peak intensity than single focus result, the cubic phase mask can help maintain a higher intensity at deeper depth. Accordingly, the cubic phase mask can help increase the penetration depth of an ultrasound imaging system.

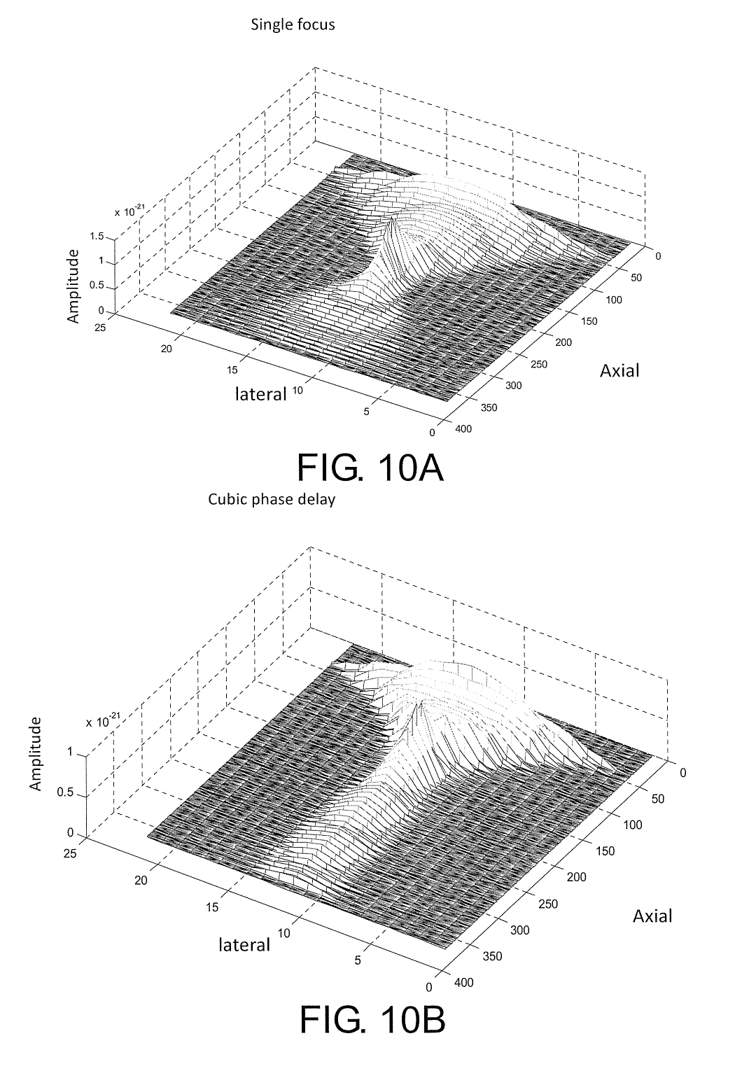

FIGS. 10A and 10B are amplitude diagrams comparing the received response of an ultrasound imaging system with and without a cubic phase mask according to an exemplary embodiment. As shown in FIGS. 10A and 10B, without cubic phase mask, an ultrasound beam diverge or spread out very quickly away from the focus. By contrast, with the cubic phase mask, the beam will not diverge quickly after passing through the focal point. Therefore, using a cubic phase mask as the asymmetric phase element can maintain the ultrasound beam intensity for a longer distance and obtain a deeper penetration depth.

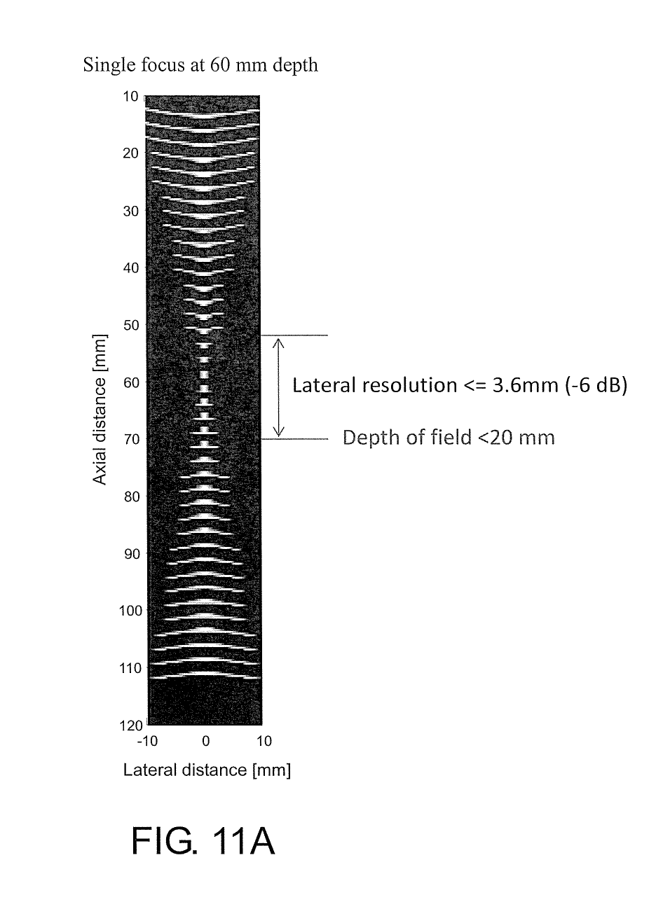

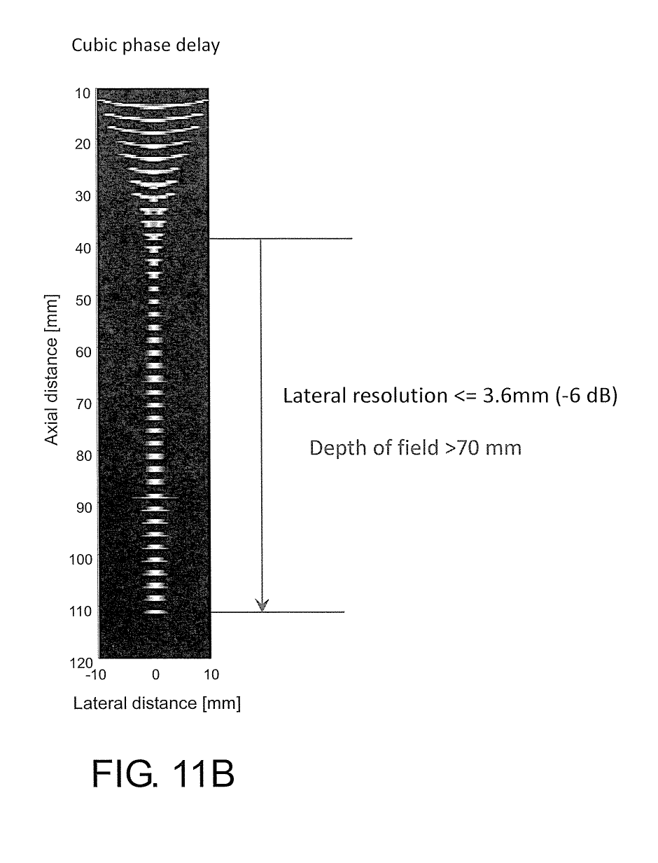

FIGS. 11A and 11B are images comparing a depth of field of an ultrasound imaging system with a single focus emission and reception and a depth of field of an ultrasound system with an added cubic phase mask to code the transmitting and receiving signals according to an exemplary embodiment. As shown in FIGS. 11A and 11B, with the cubic phase mask, the depth of field for the ultrasound imaging system in an exemplary embodiment can be extended to at least 3.5 times longer than the traditional single focal point case.

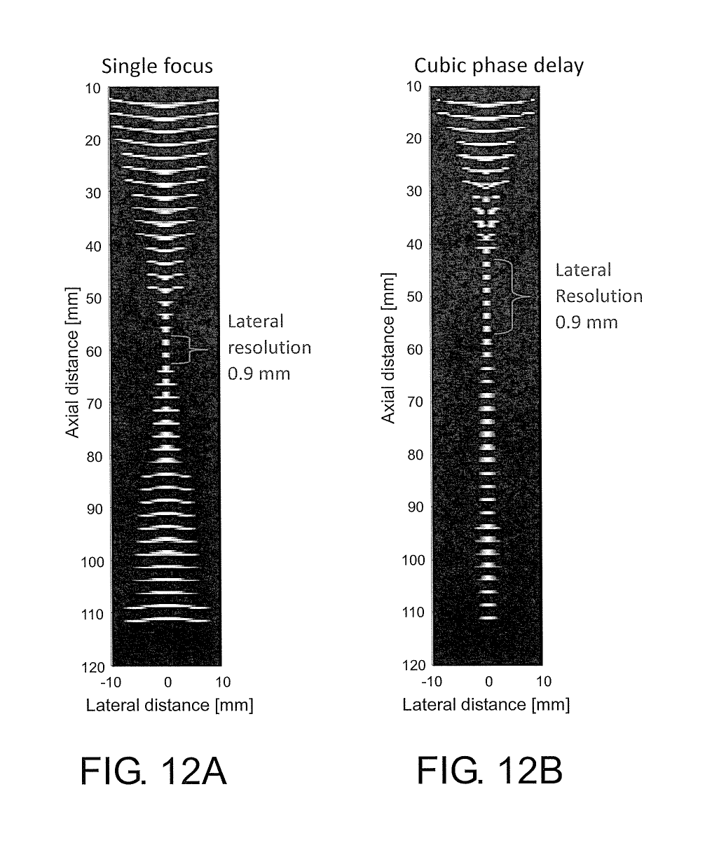

FIGS. 12A and 12B are images comparing the focal lateral resolutions of an ultrasound imaging system with and without a cubic phase mask according to an exemplary embodiment. As shown in FIGS. 12A and 12B, with the cubic phase mask, the lateral resolution around the focal point can be as fine as the single focal point case. Accordingly, the addition of the asymmetric phase element in the ultrasound imaging system can extend the depth of field without sacrificing the lateral resolution.

With reference to the foregoing description of the ultrasound imaging system 100 depicted in FIG. 1, as well as the transmitter 110 and the receiver 120 depicted in FIGS. 2A and 2B, a method for increasing a depth of field adapted for an ultrasound imaging system (e.g. the ultrasound imaging system 100) can be obtained. In the method, a plurality of energy signals coded by a first asymmetric phase element are transmitted toward an object to be imaged. Moreover, a plurality of echo signals from the object to be imaged is received. The received signals are coded with a second asymmetric phase element, and an image data set is reconstructed with an extended depth of field by decoding the received signals.

The step of transmitting the energy signals coded by the first asymmetric phase element toward the object to be imaged may include delaying the energy signals with a system time delay, coding the delayed energy signals with the first asymmetric phase element, and converting the delayed energy signals into a plurality of ultrasound signals and respectively transmitting the coded ultrasound signals toward the object to be imaged with an array transducer.

Moreover, the step of receiving the echo signals from the object to be imaged may include converting each of the echo signals into a plurality of electrical signals with an array transducer, coding the electrical signals with the second asymmetric phase element, summing the coded electrical signals into a RF signal with a signal adder, and combining the RF signals into an intermediate image and decoding the intermediate image into a decoded ultrasound image with a signal processor. In addition, the step of combining the RF signals into the intermediate image and decoding the intermediate image into the decoded ultrasound image may include combining the RF signals to form the intermediate image with a RF signal combiner, and decoding the intermediate image into the decoded ultrasound image with a decoding filter.

According to some embodiments of the disclosure, the first asymmetric phase element and the second asymmetric phase element include an asymmetric phase mask, an asymmetric phase function, an asymmetric delay time table, or an asymmetric phase surface integrated with a lens. It should also be noted that, when suitable for an application, either the step of coding the delayed energy signals with the first asymmetric phase element, or the step of coding the electrical signals with the second asymmetric phase element can be omitted.

For example, with reference to the foregoing description of the ultrasound imaging system 100 depicted in FIG. 1, as well as the transmitter 110 and the receiver 120 depicted in FIGS. 4A and 4B, a method for increasing a depth of field adapted for an ultrasound imaging system (e.g. the ultrasound imaging system 100) can be obtained. In the method, a plurality of energy signals coded by an asymmetric phase element is transmitted toward an object to be imaged. Moreover, a plurality of echo signals from the object to be imaged is received, and an image data set is reconstructed with an extended depth of field by decoding the received signals.

The step of transmitting the energy signals coded by the asymmetric phase element toward the object to be imaged may include delaying the energy signals with a system time delay, coding the delayed energy signals with the asymmetric phase element, and converting the delayed energy signals into a plurality of ultrasound signals and respectively transmitting the coded ultrasound signals toward the object to be imaged with an array transducer.

Moreover, the step of receiving the echo signals from the object to be imaged may include converting each of the echo signals into a plurality of electrical signals with an array transducer, summing the coded electrical signals into a RF signal with a signal adder, and combining the RF signals into an intermediate image and decoding the intermediate image into a decoded ultrasound image with a signal processor. In addition, the step of combining the RF signals into the intermediate image and decoding the intermediate image into the decoded ultrasound image may include combining the RF signals to form the intermediate image with a RF signal combiner, and decoding the intermediate image into the decoded ultrasound image with a decoding filter.

In another example, with reference to the foregoing description of the ultrasound imaging system 100 depicted in FIG. 1, as well as the transmitter 110 and the receiver 120 depicted in FIGS. 5A and 5B, a method for increasing a depth of field adapted for an ultrasound imaging system (e.g. the ultrasound imaging system 100) can be obtained. In the method, a plurality of energy signals is transmitted toward an object to be imaged. Moreover, a plurality of echo signals from the object to be imaged is received. The received signals are coded with an asymmetric phase element, and an image data set is reconstructed with an extended depth of field by decoding the received signals.

The step of transmitting the energy signals toward the object to be imaged may include delaying the energy signals with a system time delay, and converting the delayed energy signals into a plurality of ultrasound signals and respectively transmitting the ultrasound signals toward the object to be imaged with an array transducer.

Moreover, the step of receiving the echo signals from the object to be imaged may include converting each of the echo signals into a plurality of electrical signals with an array transducer, coding the electrical signals with the asymmetric phase element, summing the coded electrical signals into a RF signal with a signal adder, and combining the RF signals into an intermediate image and decoding the intermediate image into a decoded ultrasound image with a signal processor. In addition, the step of combining the RF signals into the intermediate image and decoding the intermediate image into the decoded ultrasound image may include combining the RF signals to form the intermediate image with a RF signal combiner, and decoding the intermediate image into the decoded ultrasound image with a decoding filter.

In view of the foregoing, exemplary embodiments in the disclosure have provided a method for increasing depth of field and an ultrasound imaging system using the same. One or more asymmetric phase elements can be added to code the transmitting and receiving signals in an ultrasound imaging system. Moreover, the asymmetric phase elements code the transmitted and received signals in such a way that the point-spread function and the system transfer function do not change appreciably as a function of misfocus. Once the image is transformed into digital form, a signal processing step decodes the image and produces the final ultrasound image with extended depth of field. Accordingly, fewer transmissions are required to construct images, and the method and ultrasound imaging system for increasing depth of field can achieve a high frame rate ultrasound image.

It will be apparent to those skilled in the art that various modifications and variations can be made to the structure of the disclosed embodiments without departing from the scope or spirit of the disclosure. In view of the foregoing, it is intended that the disclosure cover modifications and variations of this disclosure provided they fall within the scope of the following claims and their equivalents.

What is claimed is:

1. An ultrasound imaging system, comprising:
a transmitter adapted to transmit a plurality of energy signals coded by a first asymmetric phase element toward an object to be imaged; and
a receiver adapted to receive a plurality of echo signals from the object to be imaged, respectively code the received signals with a second asymmetric phase element, and reconstruct an image data set with an extended depth of field by decoding the received signals.

2. The ultrasound imaging system of claim 1, wherein the transmitter comprises:
a system time delay delaying the energy signals;
the first asymmetric phase element coding the delayed energy signals; and
an array transducer converting the delayed energy signals into a plurality of ultrasound signals and respectively transmitting the coded ultrasound signals toward the object to be imaged.

3. The ultrasound imaging system of claim 1, wherein the receiver comprises:
an array transducer converting each of the echo signals into a plurality of electrical signals;
the second asymmetric phase element coding the electrical signals;
a signal adder summing the coded electrical signals into a radio frequency (RF) signal; and
a signal processor combining the RF signals into an intermediate image and decoding the intermediate image into a decoded ultrasound image.

4. The ultrasound imaging system of claim 3, wherein the signal processor in the receiver comprises:
a RF signal combiner combining the RF signals to form the intermediate image; and
a decoding filter decoding the intermediate image into the decoded ultrasound image.

5. The ultrasound imaging system of claim 1, wherein the first asymmetric phase element and the second asymmetric phase element comprise an asymmetric phase mask, an asymmetric phase function, an asymmetric delay time table, or an asymmetric phase surface integrated with a lens.

6. An ultrasound imaging system, comprising:
a transmitter adapted to transmit a plurality of energy signals coded by an asymmetric phase element toward an object to be imaged; and
a receiver adapted to receive a plurality of echo signals from the object to be imaged and reconstruct an image data set with an extended depth of field by decoding the received signals.

7. The ultrasound imaging system of claim 6, wherein the transmitter comprises:
a system time delay delaying the energy signals;
the asymmetric phase element coding the delayed energy signals; and
an array transducer converting the delayed energy signals into a plurality of ultrasound signals and respectively transmitting the coded ultrasound signals toward the object to be imaged.

8. The ultrasound imaging system of claim 6, wherein the receiver comprises:
an array transducer converting each of the echo signals into a plurality of electrical signals;
a signal adder summing the electrical signals into a RF signal; and
a signal processor combining the RF signals into an intermediate image and decoding the intermediate image into a decoded ultrasound image.

9. The ultrasound imaging system of claim 8, wherein the signal processor in the receiver comprises:
a RF signal combiner combining the RF signals to form the intermediate image; and
a decoding filter decoding the intermediate image into the decoded ultrasound image.

10. The ultrasound imaging system of claim 6, wherein the asymmetric phase element comprises an asymmetric phase mask, an asymmetric phase function, an asymmetric delay time table, or an asymmetric phase surface integrated with a lens.

11. An ultrasound imaging system, comprising:
a transmitter adapted to transmit a plurality of energy signals toward an object to be imaged; and
a receiver adapted to receive a plurality of echo signals from the object to be imaged, respectively code the received signals with an asymmetric phase element, and reconstruct an image data set with an extended depth of field by decoding the received signals.

12. The ultrasound imaging system of claim 11, wherein the transmitter comprises:
a system time delay delaying the energy signals; and
an array transducer converting the delayed energy signals into a plurality of ultrasound signals and respectively transmitting the ultrasound signals toward the object to be imaged.

13. The ultrasound imaging system of claim 11, wherein the receiver comprises:
an array transducer converting each of the echo signals into a plurality of electrical signals;
the asymmetric phase element coding the electrical signals;
a signal adder summing the coded electrical signals into a RF signal; and
a signal processor combining the RF signals into an intermediate image and decoding the intermediate image into a decoded ultrasound image.

14. The ultrasound imaging system of claim 13, wherein the signal processor in the receiver comprises:
a RF signal combiner combining the RF signals to form the intermediate image; and
a decoding filter decoding the intermediate image into the decoded ultrasound image.

15. The ultrasound imaging system of claim 11, wherein the asymmetric phase element comprises an asymmetric phase mask, an asymmetric phase function, an asymmetric delay time table, or an asymmetric phase surface integrated with a lens.

16. A method for an ultrasound imaging system, the method comprising:
transmitting a plurality of energy signals coded by a first asymmetric phase element toward an object to be imaged; and
receiving a plurality of echo signals from the object to be imaged, respectively coding the received signals with a second asymmetric phase element, and reconstructing an image data set with an extended depth of field by decoding the received signals.

17. The method of claim 16, wherein the step of transmitting the energy signals coded by the first asymmetric phase element toward the object to be imaged comprises:
delaying the energy signals with a system time delay;
coding the delayed energy signals with the first asymmetric phase element; and
converting the delayed energy signals into a plurality of ultrasound signals and respectively transmitting the coded ultrasound signals toward the object to be imaged with an array transducer.

18. The method of claim 16, wherein the step of receiving the echo signals from the object to be imaged comprises:
converting each of the echo signals into a plurality of electrical signals with an array transducer;
coding the electrical signals with the second asymmetric phase element;
summing the coded electrical signals into a RF signal with a signal adder; and
combining the RF signals into an intermediate image and decoding the intermediate image into a decoded ultrasound image with a signal processor.

19. The method of claim 18, wherein the step of combining the RF signals into the intermediate image and decoding the intermediate image into the decoded ultrasound image comprises:
combining the RF signals to form the intermediate image with a RF signal combiner; and
decoding the intermediate image into the decoded ultrasound image with a decoding filter.

20. The method of claim 16, wherein the first asymmetric phase element and the second asymmetric phase element comprise an asymmetric phase mask, an asymmetric phase function, an asymmetric delay time table, or an asymmetric phase surface integrated with a lens.

21. A method for an ultrasound imaging system, the method comprising:
transmitting a plurality of energy signals coded by an asymmetric phase element toward an object to be imaged; and
receiving a plurality of echo signals from the object to be imaged and reconstruct an image data set with an extended depth of field by decoding the received signals.

22. The method of claim 21, wherein the step of transmitting the energy signals coded by the asymmetric phase element toward the object to be imaged comprises:
delaying the energy signals with a system time delay;
coding the delayed energy signals with the asymmetric phase element; and
converting the delayed energy signals into a plurality of ultrasound signals and respectively transmitting the coded ultrasound signals toward the object to be imaged with an array transducer.

23. The method of claim 21, wherein the step of receiving the echo signals from the object to be imaged comprises:
converting each of the echo signals into a plurality of electrical signals with an array transducer;
summing the electrical signals into a RF signal with a signal adder; and
combining the RF signals into an intermediate image and decoding the intermediate image into a decoded ultrasound image with a signal processor.

24. The method of claim 23, wherein the step of combining the RF signals into the intermediate image and decoding the intermediate image into the decoded ultrasound image comprises:
combining the RF signals to form the intermediate image with a RF signal combiner; and
decoding the intermediate image into the decoded ultrasound image with a decoding filter.

25. The method of claim 21, wherein the asymmetric phase element comprises an asymmetric phase mask, an asymmetric phase function, an asymmetric delay time table, or an asymmetric phase surface integrated with a lens.

26. A method for an ultrasound imaging system, the method comprising:
transmitting a plurality of energy signals toward an object to be imaged; and
receiving a plurality of echo signals from the object to be imaged, respectively code the received signals with an asymmetric phase element, and reconstruct an image data set with an extended depth of field by decoding the received signals.

27. The method of claim 26, wherein the step of transmitting the energy signals toward the object to be imaged comprises:
delaying the energy signals with a system time delay; and
converting the delayed energy signals into a plurality of ultrasound signals and respectively transmitting the ultrasound signals toward the object to be imaged with an array transducer.

28. The method of claim 26, wherein the step of receiving the echo signals from the object to be imaged comprises:
converting each of the echo signals into a plurality of electrical signals with an array transducer;
coding the electrical signals with the asymmetric phase element;
summing the coded electrical signals into a RF signal with a signal adder; and
combining the RF signals into an intermediate image and decoding the intermediate image into a decoded ultrasound image a signal processor.

29. The method of claim 28, wherein the step of combining the RF signals into the intermediate image and decoding the intermediate image into the decoded ultrasound image comprises:
combining the RF signals to form the intermediate image with a RF signal combiner; and
decoding the intermediate image into the decoded ultrasound image with a decoding filter.

30. The method of claim 26, wherein the asymmetric phase element comprises an asymmetric phase mask, an asymmetric phase function, an asymmetric delay time table, or an asymmetric phase surface integrated with a lens.
